(19) United States
(12) Patent Application Publication
(10) Pub. No.: US 2020/0170838 A1
(43) Pub. Date: Jun. 4, 2020

(54) EYE WEARING DEVICE

(71) Applicant: Industrial Technology Research Institute, Hsinchu (TW)

(72) Inventors: Yun-Chung Teng, Kaohsiung City (TW); Yu-Bing Liou, Hsinchu City (TW); Hsin-Yi Hsu, Taoyuan City (TW); Ying-Wen Shen, Miaoli County (TW); Sen-Lu Chen, Miaoli County (TW); Yu-Chi Wang, New Taipei City (TW); Hsin-Hsin Shen, Taipei City (TW)

(73) Assignee: Industrial Technology Research Institute, Hsinchu (TW)

(21) Appl. No.: 16/683,277

(22) Filed: Nov. 14, 2019

Related U.S. Application Data
(60) Provisional application No. 62/760,929, filed on Nov. 14, 2018.

(51) Int. Cl. A61F 9/00

(52) U.S. Cl. CPC A61F 9/0017

(57) ABSTRACT

An eye wearing device is provided, including a body, at least one reservoir and at least one microchannel. The body has a first surface, a second surface, a center, a first outer edge of the first surface and a second outer edge of the second surface. The reservoir is disposed in the body to load the drug. The microchannel is disposed near the first outer edge of the first surface and the second outer edge of the second surface. One end of the microchannel is connected to the reservoir for filling the drug into the reservoir. The other end of the microchannel is an opening facing an edge of the eye wearing device for contacting the cornea and/or the sclera, and the opening is connected to the first outer edge of the first surface and the second outer edge of the second surface.

### CROSS-REFERENCE TO RELATED APPLICATION

This application claims the priority benefits of U.S. provisional application Ser. No. 62/760,929, filed on Nov. 14, 2018. The entirety of the above-mentioned patent applications is hereby incorporated by reference herein and made a part of specification.

### TECHNICAL FIELD

The disclosure relates to an eye wearing device, in particular to an eye wearing device for drug delivery and a use method thereof.

### BACKGROUND

The existing methods for improving bioavailability of eyedrops mainly include drug/dosage form development and implantation/wearing drug delivery. However, for a slow release composite medical material, specific categories of drugs are generally made into nanoparticles, hydrogels or polymer carriers, and needs to be bound with a specific drug, and therefore the storage and the manufacturing processes are restricted to the drug, and it is not applicable for personalized drug. Additionally, the problem of excessively fast release often occurs in known technologies. For storage volume or bioavailability improvement, a good fixing and oxygen permeating design is needed for the connection with the eye surface. Meanwhile, there are many limitations in drug selection and matching in the known art, and the range of currently available excipients, pH value and osmotic pressure is still very narrow even for eyedrops. Clinically, the existing medical material for drug delivery also faces the problems of low bioavailability and poor drug compliance. Therefore, the development of an eye wearing device capable of improving the drug release condition, realizing simple and convenient operation and flexibly matched with personalized drugs is in urgent need.

### SUMMARY

The disclosure provides an eye wearing device capable of being used for storing, slowly releasing and supplementing a treatment drug without hindering the vision of a user.

The eye wearing device of the disclosure includes a body, at least one reservoir and at least one microchannel. The body includes a first surface, a second surface, a center, a first outer edge of the first surface and a second outer edge of the second surface. The at least one reservoir is arranged in the body and is configured to load a drug. The at least one microchannel is arranged in a position close to the first outer edge of the first surface and the second outer edge of the second surface. One end of the microchannel is connected to the reservoir so as to fill the drug into the reservoir. The other end of the microchannel is an opening facing the edge of the eye wearing device and is configured to be in contact with a cornea and/or a sclera. The opening is connected to the first outer edge of the first surface and the second outer edge of the second surface. The average diameter of the cross section of the microchannel is smaller than or equal to the average diameter of the cross section of the reservoir. The average curvature radius of the eye wearing device is 6 mm to 15 mm. The eye wearing device is worn on the cornea and/or the sclera of the user through the second surface.

Based on the above, the eye wearing device of the disclosure includes at least one reservoir and at least one microchannel, in which the microchannel can eliminate stoppers such as bubbles and promote drug filling or supplementation, and can further be matched with material properties so as to enhance the drug supplementing capability through blinking. The microchannel is subjected to specific surface treatment so as to enhance the efficiency. Therefore, the eye wearing device of the disclosure can be used for effectively treating eye diseases in an auxiliary way, is particularly favourable for the use of personalized drugs, and can be used for storing, slowly releasing and supplementing the treatment drug without hindering the vision of the user.

To make the features and advantages of the disclosure clear and easy to understand, the following gives a detailed description of embodiments with reference to accompanying drawings.

### BRIEF DESCRIPTION OF THE DRAWINGS

FIG. 1A is a top view schematic diagram of an eye wearing device according to the first embodiment of the disclosure. FIG. 1B is a straight cutting cross-section schematic diagram along a tangent line A-A′ in FIG. 1A. FIG. 1C is a straight cutting cross-section schematic diagram along a tangent line B-B′ in FIG. 1A.

FIG. 2 is a top view schematic diagram of an eye wearing device according to the second embodiment of the disclosure.

FIG. 3A to FIG. 3B are stereoscopic schematic diagrams of an eye wearing device according to the disclosure.

FIG. 4A to FIG. 4C are stereoscopic schematic diagrams of another eye wearing device according to the disclosure.

FIG. 5A to FIG. 5B are schematic diagrams of a configuration mode of a reservoir and a microchannel in an eye wearing device according to the third embodiment of the disclosure, in which FIG. 5A is a top view schematic diagram, and FIG. 5B is a straight cutting cross-section schematic diagram along a tangent line C-C′ in FIG. 5A.

FIG. 6A to FIG. 6C are schematic diagrams of a configuration mode of a reservoir and a microchannel in an eye wearing device according to the fourth embodiment of the disclosure, in which FIG. 6A is a top view schematic diagram, and FIG. 6B and FIG. 6C are straight cutting cross-section schematic diagrams along a tangent line D-D′ in FIG. 6A. FIG. 6B and FIG. 6C respectively represent different modes of a gradually reducing microchannel. FIG. 6B is a smooth gradually reducing mode. FIG. 6C is a stepped gradually reducing mode.

FIG. 7A to FIG. 7D are schematic diagrams of a package design of an eye wearing device according to the disclosure, in which FIG. 7A is a cross-section schematic diagram, FIG. 7B is a top view schematic diagram, FIG. 7C is a stereoscopic schematic diagram, and FIG. 7D is a local amplification schematic diagram.

FIG. 8A, FIG. 8B and FIG. 8C are schematic diagrams of another package design of the eye wearing device according to the disclosure.

FIG. 9A and FIG. 9B are schematic diagrams of yet another package design of the eye wearing device according to the disclosure, in which FIG. 9A is a stereoscopic schematic diagram, and FIG. 9B is a cross-section schematic diagram.

### DETAILED DESCRIPTION OF DISCLOSED EMBODIMENTS

The following embodiments are described in details with reference to accompanying drawings, but the provided embodiments are not intended to limit the scope covered by the present disclosure. In addition, the drawings are drawn only for the purpose of description, and are not drawn according to original sizes. For ease of understanding, same elements in the following description are described by using the same signs. Terms such as “includes”, “comprises”, and “having” used herein are all inclusive terms, namely, mean “includes but not limited to”. In addition, the directional terms mentioned herein, like “above” and “below”, are only used to refer to the directions in the accompanying drawings and are not intended to limit the disclosure. In addition, the quantities and shapes mentioned in the specification are only used to specifically describe the disclosure to facilitate understanding of contents of the disclosure, and are not intended to limit the disclosure.

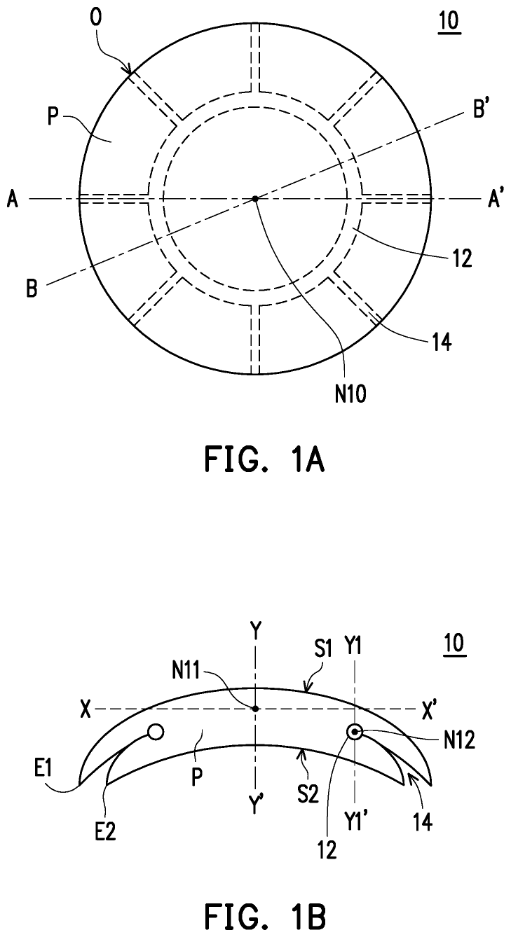

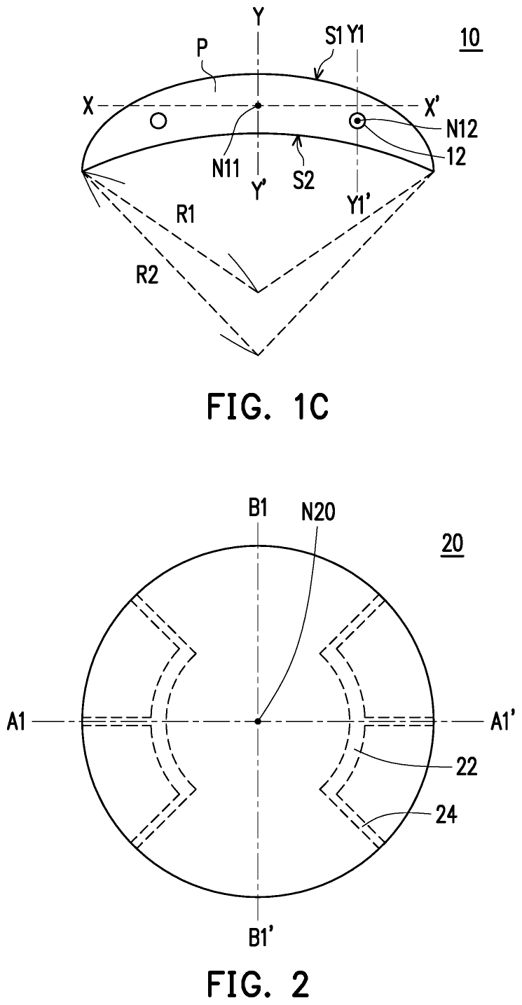

FIG. 1A is a top view schematic diagram of an eye wearing device according to the first embodiment of the disclosure. FIG. 1B is a straight cutting cross-section schematic diagram along a tangent line A-A′ in FIG. 1A. FIG. 1C is a straight cutting cross-section schematic diagram along a tangent line B-B′ in FIG. 1A.

Referring to FIG. 1A, FIG. 1B and FIG. 1C altogether, an eye wearing device 10 can include a body P, at least one reservoir 12 and at least one microchannel 14. The body P can structurally include a first surface S1, a second surface S2, a center N10 and N11, an outer edge E1 of the first surface S1 and an outer edge E2 of the second surface S2. The above-mentioned center N10 refers to a center point of the body P defined by the top view schematic diagram of FIG. 1A (can be defined by an intersection point of the tangent line A-A′ and the tangent line B-B′ in FIG. 1A), and the center N11 refers to a center point of the body P defined by the cross-section schematic diagrams of FIG. 1B and FIG. 1C. Referring to FIG. 1B and FIG. 1C, Y-Y′ is a longitudinal axis line penetrating through the body P through the center N11 in a longitudinal direction, X-X′ is a horizontal axis line penetrating through the body P through the center N11 in a horizontal direction, and the longitudinal axis line Y-Y′ and the horizontal axis line X-X′ are vertical to each other.

The average curvature radius of the eye wearing device 10 can be about 6 mm to 15 mm, and for example, can be 6 mm to 14.5 mm, 6.5 mm to 13 mm, 6.5 mm to 12 mm, 7 mm to 9 mm, 8.5 mm to 10.5 mm, 7 mm to 10 mm, etc., but the disclosure is not limited thereto. Further, referring to FIG. 1C, the first surface S1 and the second surface S2 of the eye wearing device 10 can respectively have different average curvature radii R1 and R2, and the first average curvature radius R1 of the first surface S1 is smaller than the second average curvature radius R2 of the second surface S2. For example, the first average curvature radius R1 can be about 6.0 mm to 14.8 mm, for example, 6 mm to 14.5 mm, 6 mm to 13 mm, 6.5 mm to 12 mm, 7 mm to 11.5 mm, 7.5 mm to 11 mm, 8 mm to 10.5 mm, 7 mm to 10 mm, etc., but the disclosure is not limited thereto. The second average curvature radius R2 can be about 6.2 mm to 15.0 mm, for example, 6.5 mm to 14.5 mm, 6.5 mm to 13 mm, 7 mm to 12.5 mm, 7 mm to 12 mm, 7.5 mm to 11.5 mm, 8 mm to 11 mm, 8 mm to 10 mm, etc., but the disclosure is not limited thereto. Additionally, the eye wearing device 10 uses the second surface S2 to be in contact with the cornea and/or the sclera of the user when being worn.

Additionally, a material of the eye wearing device 10 can include bio-derived polymers, non-bio-derived polymers or a combination thereof, in which the bio-derived polymers can include collagen, gelatin, chitin, cellulose or a combination thereof, but the disclosure is not limited thereto. The non-bio-derived polymers can include polyethylene glycol (PEG), propylene glycol diacrylate (PPGDA), polydimethylsiloxane (PDMS), poly(methyl methacrylate) (PMMA), poly(hydroxyethyl methacrylate) (PHEMA) or a combination thereof, but the disclosure is not limited thereto.

The reservoir 12 is arranged in the body P, and can be configured to load the drug. The microchannel 14 is arranged in the position close to the outer edge E1 of the first surface S1 and the outer edge E2 of the second surface S2, and can be configured to fill the drug into the reservoir 12. The reservoir 12 and the microchannel 14 can further be subjected to hydrophilic or anti-sticking modification treatment on the surface, so that drug filling or bubble elimination is promoted through capillary action, and material properties (such as softness and elasticity) can be further matched so as to enhance the drug supplementing capability through blinking. The hydrophilic modification treatment mode can include the steps that hydrophilic polymers, ionic functional groups or interface active agents are mixed into a substrate, are immersed/coated onto the surface of the substrate or are grafted onto the surface of the substrate through chemical reaction, the surface of a substance can also be subjected to oxidization, crosslinking, easy-to-react functional group addition or micro structure change treatment by using plasma, ultraviolet light, heat treatment or other reactant gas, but the disclosure is not limited thereto. In detail, the hydrophilic polymers can include polyacrylamide (PAM or PAAM), polyethylene glycol (PEG), etc., the ionic functional groups can include primary/secondary amine, carboxylate radicals, etc., and the interface active agents can include sodium dodecyl sulfate (SDS), polyethylene glycol p-(1,1,3,3-tetramethylbutyl)-phenyl ether (Triton X-100), {[3-(Dodecanoylamino)propyl](dimethyl)ammonio}acetate, etc., but the disclosure is not limited thereto. The mode of the anti-sticking modification treatment is similar to that of the hydrophilic modification, and is not repeated herein, but the disclosure is not limited thereto.

In the present embodiment, the reservoir 12 can be in a ring shape, an arc shape, a line shape or a combination thereof, but the disclosure is not limited thereto. The reservoir 12 is designed to be in a position not hindering the vision in the eye wearing device 10. Referring to FIG. 1B and FIG. 1C, Y1-Y1′ is a longitudinal axis line penetrating through the body P through the center N12 of the reservoir 12 in a longitudinal direction. For example, the center N12 of the reservoir 12 can be arranged in a position 4 mm to 14 mm away from the center N11 of the body, the center N12 of the reservoir 12 can also be arranged in a position about 4.5 mm to 14 mm, 6 mm to 14 mm, 4 mm to 12 mm, 8 mm to 12 mm, 10 mm to 12 mm, 6.5 mm to 10 mm, 8 mm to 10 mm, 7 mm to 11 mm, 5.5 mm to 10.5 mm, 4.5 mm to 8 mm away from the center N11 of the body, but the disclosure is not limited thereto. In other words, the shape of the reservoir 12 and the arrangement position of the reservoir 12 in the eye wearing device 10 can be adjusted according to practical requirements.

In the present embodiment, one end of the microchannel 14 can be connected to the reservoir 12, the other end faces an opening O arranged at the edge of the eye wearing device 10, and is configured to be in contact with the cornea and/or the sclera, and the opening O is connected to the outer edge E1 of the first surface S1 and the outer edge E2 of the second surface S2. The configuration mode of the microchannel 14 can be adjusted according to practical requirements. For example, the configuration mode of the microchannel 14 in the eye wearing device 10 can include but is not limited to a radial type. Although FIG. 1A shows eight microchannels 14, the configuration number of the microchannel 14 of the disclosure is not limited thereto, and can be adjusted to be 16, 14, 12, 10 or 6, etc. according to practical requirements.

Additionally, a lubricating material, such as mucoprotein, polyethylenimine, polyethylene glycol, polyacrylic acid, polymethacrylic acid, polyitaconic acid, polymaleic acid, carboxymethyl cellulose (CMC), hydroxypropyl methylcellulose (HPMC), polyvinylpyrrolidone, polyacrylamide, poleyvinylalcohol, hyaluronic acid, dextran, poly 2-hydroxyethyl methacrylate (poly HEMA), poly sulfonates, polylactate, urea, phosphoryl choline or a combination thereof can be further coated on the surface of the eye wearing device 10. In addition, the lubricating material can also be hydrophilic polypeptides, for example, 75 or above weight percent of amino acids of the polypeptides are selected from the group consisting of aspartic acid (Asp or D), glutamic acid (Glu or E), histidine (His or H), lysine (Lys or K), asparagine (Asn or N), glutamine (Gln or Q), arginine (Arg or R), serine (Ser or S), threonine (Thr or T) and tyrosine (Tyr or Y), but the disclosure is not limited thereto. The material is mainly directed to improve wearing comfort, and can avoid moisture evaporation.

Referring to FIG. 1A, FIG. 1B and FIG. 1C altogether, the width of the reservoir 12 can be 30 μm to 5 mm, for example, 30 μm to 50 μm, 50 μm to 70 μm, 70 μm to 100 μm, 100 μm to 4.5 mm, 500 μm to 4.5 mm, 1 mm to 4 mm, 1.5 mm to 4 mm, 1.5 mm to 3.5 mm, 2 mm to 3.5 mm, 2.5 mm to 3.5 mm, 2.5 mm to 3 mm, etc., but the disclosure is not limited thereto. The height of the reservoir 12 can be 10 μm to 200 μm, for example, 20 μm to 180 μm, 30 μm to 150 μm, 50 μm to 120 μm, 50 μm to 100 μm, 60 μm to 80 μm, etc., but the disclosure is not limited thereto. The total volume of the drug loaded in the reservoir 12 can be 0.002 μL to 20 μL, for example, 0.05 μL to 20 μL, 0.1 μL to 20 μL, 0.5 μL to 20 μL, 1 μL to 20 μL, 2 μL to 20 μL, 5 μL to 20 μL, 10 μL to 20 μL, 5 μL to 15 μL, 5 μL to 10 μL, etc., but the disclosure is not limited thereto.

Additionally, the average diameter of the cross section of the microchannel 14 can be 20 μm to 150 μm, for example, 30 μm to 150 μm, 50 μm to 150 μm, 50 μm to 120 μm, 50 μm to 100 μm, 50 μm to 80 μm, 80 μm to 120 μm, 100 μm to 150 μm, etc., but the disclosure is not limited thereto. The average diameter of the cross section of the microchannel 14 can be smaller than or equal to the average diameter of the cross section of the reservoir 12, in which the diameter of one end of the microchannel 14 connected to the reservoir 12 can be 10 μm to 200 μm, for example, 10 μm to 20 μm, 20 μm to 50 μm, 50 μm to 100 μm, 100 μm to 150 μm, 80 μm to 200 μm, etc., but the disclosure is not limited thereto. The diameter of one end of the microchannel in contact with the cornea and/or the sclera can be 40 μm to 200 μm, for example, 50 μm to 180 μm, 60 μm to 160 μm, 70 μm to 150 μm, 80 μm to 140 μm, 90 μm to 130 μm, 100 μm to 120 μm, etc., but the disclosure is not limited thereto.

Referring to FIG. 1A, FIG. 1B and FIG. 1C altogether, the appearance of the eye wearing device 10 can include but is not limited to a round shape. The diameter can be about 12 mm to 20 mm, for example, 12 mm to 15 mm, 15 mm to 18 mm, 18 mm to 20 mm, 12 mm to 18 mm, 15 mm to 20 mm, 13 mm to 15 mm, etc., but the disclosure is not limited thereto. The average thickness of the eye wearing device can be about 20 μm to 400 μm, for example, 30 μm to 350 μm, 50 μm to 300 μm, 50 μm to 250 μm, 80 μm to 320 μm, 100 μm to 300 μm, 150 μm to 300 μm, 150 μm to 200 μm, etc., but the disclosure is not limited thereto.

FIG. 2 is a top view schematic diagram of an eye wearing device according to the second embodiment of the disclosure. The second embodiment in FIG. 2 is similar to the first embodiment in FIG. 1A, so that the specifications and configuration of identical assemblies are not repeated herein.

In the second embodiment in the FIG. 2, a center N20 refers to a center point of a body defined by the top view schematic diagram of FIG. 2 (can be defined by an intersection point of a tangent line A-A′ and a tangent line B-B′ in FIG. 2). The difference between FIG. 2 and the first embodiment is that a reservoir 22 in the second embodiment is in an arc shape, for example, in a circular arc shape, and is configured by using the center N20 of an eye wearing device 20 as the center. In other words, in the first embodiment in FIG. 1A, the eye wearing device includes a ring-shaped reservoir 12, and in the second embodiment in FIG. 2, the eye wearing device includes two arc-shaped reservoirs 22, but the disclosure is not limited thereto. The design is directed to not hindering the vision. The shapes, the configuration positions and the configuration number of the reservoirs 22 can be adjusted according to practical requirements. As shown in FIG. 2, the reservoirs 22 can be symmetrically configured by using the center N20 of the eye wearing device 20 as the symmetrical center, but the disclosure is not limited thereto. The reservoirs 22 can also be in an unsymmetrical configuration mode.

As shown in FIG. 2, one end of a microchannel 24 is connected to the reservoir 22, the other end is an opening facing the edge of the eye wearing device, and can include but is not limited to radial configuration. In addition, FIG. 2 shows six microchannels 24, and each reservoir 22 is respectively connected to three microchannels 24, but the disclosure is not limited thereto, and the configuration number of the microchannels 24 can be adjusted according to practical requirements. For example, each reservoir 22 can also be connected to two, four, five, six and other number of microchannels 24, but the disclosure is not limited thereto. In addition, as mentioned above, the reservoir 22 can also be in an unsymmetrical configuration mode. For example, one reservoir 22 can be matched with two microchannels 24, and the other reservoir 22 can be matched with three microchannels 24. In other words, the numbers of the microchannels 24 matched with the two reservoirs 22 can also be different.

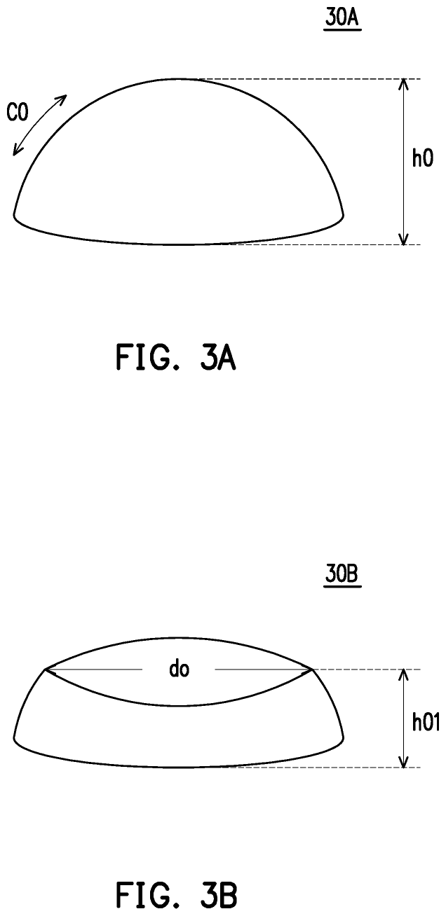

FIG. 3A to FIG. 3B are stereoscopic schematic diagrams of the eye wearing device according to the disclosure.

Referring to FIG. 3A, an eye wearing device 30A can be in a full lens type. The average curvature radius of a curve surface C0 can be about 6 mm to 15 mm, for example, 6 mm to 12 mm, 6 mm to 10 mm, 7 mm to 14 mm, 7 mm to 12 mm, 8 mm to 12 mm, 8 mm to 10 mm, etc., but the disclosure is not limited thereto. Additionally, the integral height from the top to the bottom of the full lens type eye wearing device 30A can be about 0.3 mm to 6.2 mm, for example, 0.5 mm to 3 mm, 1 mm to 4.5 mm, 2 mm to 5 mm, 2 mm to 4.5 mm, 2.5 mm to 5 mm, 4 mm to 6 mm, etc., but the disclosure is not limited thereto. Referring to FIG. 3B, an eye wearing device 30B can also be in an outer ring shape, in which the diameter d0 of a ring-shaped opening can be about 6 mm to 13 mm, for example, 6 mm to 12 mm, 7 mm to 12 mm, 8 mm to 12 mm, 8 mm to 10 mm, etc., but the disclosure is not limited thereto. Additionally, the height hOl of the ring shape can be about 0.2 mm to 6 mm, for example, 0.2 mm to 2 mm, 0.5 mm to 5 mm, 1 mm to 4.5 mm, 1.5 mm to 4 mm, 2 mm to 3.5 mm, 2.5 mm to 3 mm, 3 mm to 5 mm, etc., but the disclosure is not limited thereto. In detail, if the full lens type eye wearing device 30A in FIG. 3A is cut at a position of height hOl of the FIG. 3B, the outer ring-shaped eye wearing device 30B in FIG. 3B can be formed.

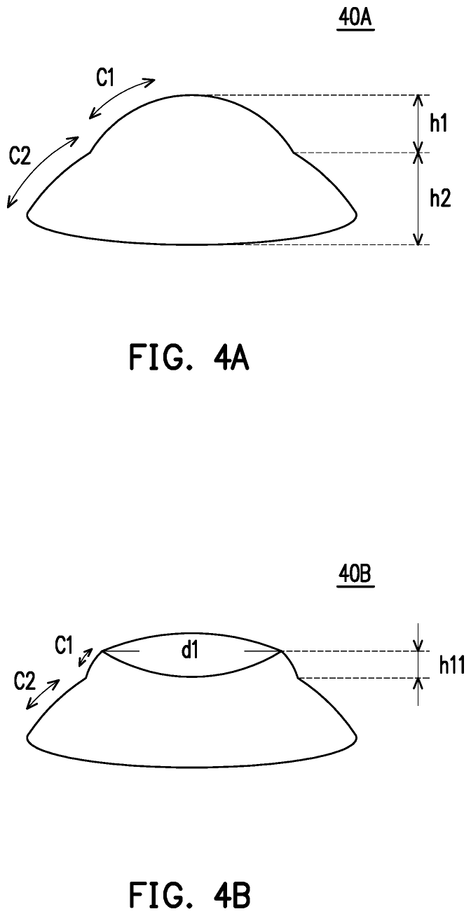

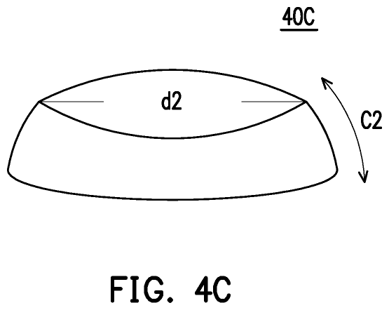

FIG. 4A to FIG. 4C are stereoscopic schematic diagrams of another eye wearing device according to the disclosure.

Referring to FIG. 4A to FIG. 4C, eye wearing devices 40A, 40B and 40C can be of a sclera-contact wearing type. Referring to FIG. 4A, the eye wearing device 40A can be of a sclera-contact wearing full lens type, and has two layers of curve surfaces C1 and C2. The curvature radius of the curve surface C1 can be about 6 mm to 9 mm, for example, 6.5 mm to 8.5 mm, 7 mm to 8.5 mm, 7.5 mm to 9 mm, etc., but the disclosure is not limited thereto. The curvature radius of the curve surface C2 can be about 7 mm to 15 mm, for example, 8 mm to 14 mm, 8 mm to 12 mm, 10 mm to 12 mm, etc., but the disclosure is not limited thereto. The height h1 of the lens with the curve surface C1 can be about 0.3 mm to 6.2 mm, for example, 0.5 to 2 mm, 1 mm to 6 mm, 2 mm to 5 mm, 3 mm to 4 mm, etc., but the disclosure is not limited thereto. The height h2 of the lens with the curve surface C2 can be about 0.2 mm to 10 mm, for example, 0.2 mm to 3 mm, 1 mm to 9 mm, 2 mm to 8 mm, 3 mm to 7 mm, 4 mm to 6 mm, etc., but the disclosure is not limited thereto.

Referring further to FIG. 4B and FIG. 4C, the eye wearing devices 40B and 40C can also be of an outer ring form of the sclera-contact wearing type. In detail, if the full lens type eye wearing device 40A in FIG. 4A is cut at a position of height h11 of the outer ring with the curve surface C1 in FIG. 4B, the outer ring type eye wearing device 40B in FIG. 4B can be formed. If the full lens type eye wearing device 40A in FIG. 4A is cut in a juncture position of a part with the curve surface C1 and a part with the curve surface C2 to cut off the part with the curve surface C1, and the outer ring type eye wearing device 40C in FIG. 4C can be formed. As shown in FIG. 4B, the diameter d1 in a ring-shaped opening can be about 6 mm to 13 mm, for example, 6 mm to 12 mm, 7 mm to 12 mm, 8 mm to 12 mm, 8 mm to 10 mm, etc., but the disclosure is not limited thereto. Additionally, the height h11 of the outer ring of the part with the curve surface C1 can be about 0.2 mm to 5 mm, for example, 0.5 mm to 4.5 mm, 1 mm to 4 mm, 1.5 mm to 3.5 mm, 2 mm to 3.5 mm, 2.5 mm to 3 mm, 3 mm to 5 mm, etc., but the disclosure is not limited thereto. As shown in FIG. 4C, the diameter d2 of the ring-shaped opening can be about 9 mm to 19 mm, for example, 9 mm to 11 mm, 10 mm to 18 mm, 10.5 mm to 17.5 mm, 11 mm to 17 mm, 12 mm to 16 mm, 12.5 mm to 15.5 mm, 13 mm to 15 mm, etc., but the disclosure is not limited thereto.

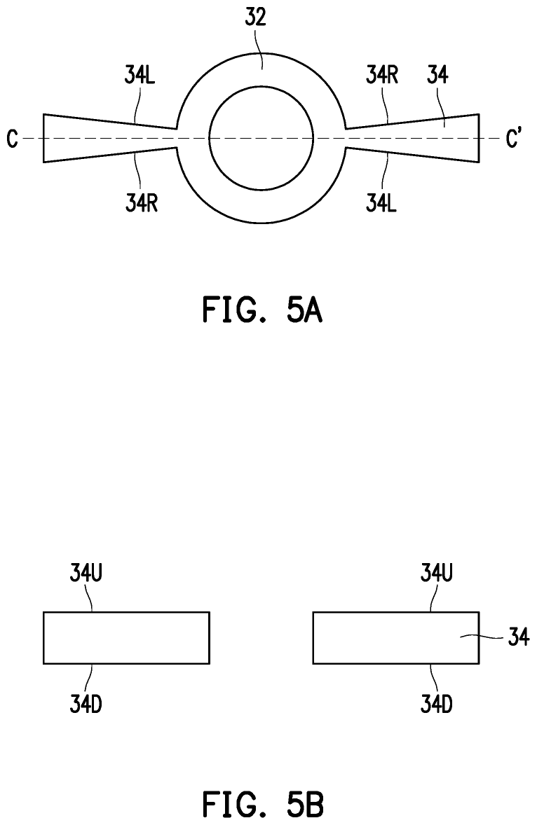

FIG. 5A to FIG. 5B are schematic diagrams of a configuration mode of a reservoir and a microchannel in an eye wearing device according to the third embodiment of the disclosure, in which FIG. 5A is a top view schematic diagram, and FIG. 5B is a straight cutting cross-section schematic diagram along a tangent line C-C′ in FIG. 5A. The third embodiment in FIG. 5A and FIG. 5B is similar to the first embodiment of FIG. 1A, so that the specifications and the configuration of identical assemblies are not repeated herein. It should be noted that, in order to clearly show the mode configuration of a reservoir 32 and microchannels 34, the drawing of an edge contour line of the eye wearing device is omitted in FIG. 5A and FIG. 5B, and only the reservoir 32 and the microchannels 34 are drawn. Additionally, only two microchannels 34 are drawn in this part to be connected to the reservoir 32, but the number of the microchannels 34 can still be adjusted according to practical requirements, and the disclosure is not limited thereto. For example, two, four, six, eight, ten and other number of microchannels 34 can be arranged to be connected to the reservoir 32.

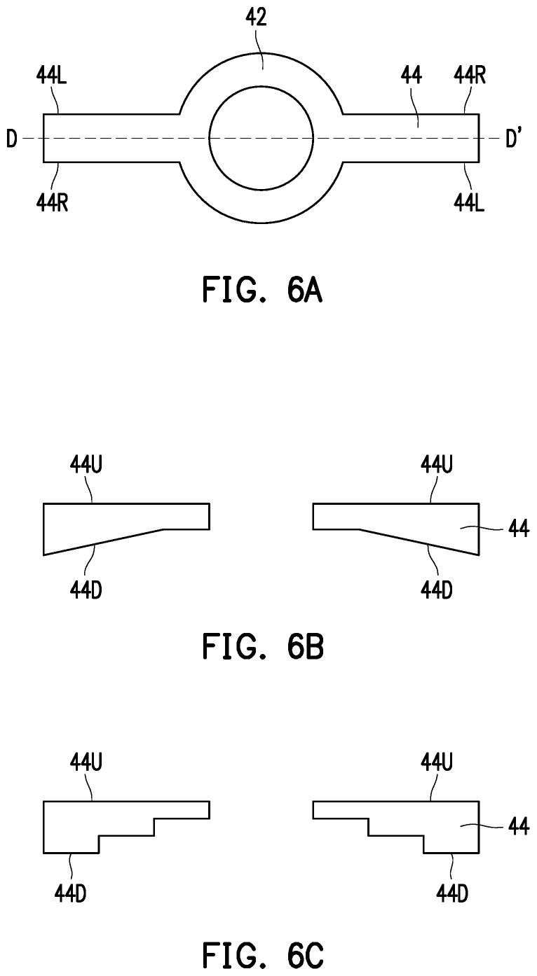

Referring to FIG. 5A and FIG. 5B, the diameter of the microchannel 34 is gradually reduced from the edge of the eye wearing device to the reservoir 32 so as to enhance a drug filling and bubble eliminating mechanism promoted through capillary action. In the present embodiment, the diameter of the microchannel 34 can be gradually reduced from the edge of the eye wearing device in the top view direction of the eye wearing device to the reservoir 32. Therefore, in the top view schematic diagram of FIG. 5A, the diameter of the microchannel 34 is gradually reduced from the edge of the eye wearing device to the reservoir 32. In the cross-section schematic diagram of FIG. 5B, the diameters of the microchannels 34 are uniform. More specifically, through the top view schematic diagram of FIG. 5A, if an angle from one end, near the edge of the eye wearing device, of the microchannel 34 to the reservoir 32 is used as an inspection view angle, two side walls of the microchannels 34 are respectively defined as a left side wall 34L and a right side wall 34R. As shown in FIG. 5A, the left side wall 34L and the right side wall 34R of the microchannel 34 are in a mutually and gradually approaching mode. In the cross-section schematic diagram of FIG. 5B, the distance between an upper side wall 34U and a lower side wall 34D of the microchannel 34 keeps unchanged. In other words, in the present embodiment, the width between the left side wall 34L and the right side wall 34R of the microchannel 34 is gradually reduced from the edge of the eye wearing device to the reservoir 32, but the height between the upper side wall 34U and the lower side wall 34D of the microchannel 34 keeps unchanged from the edge of the eye wearing device to the reservoir 32. The disclosure is not limited thereto. For example, the diameter of the microchannel 34 can also be gradually reduced from the edge of the eye wearing device to the reservoir 32 in the cross-sectional direction of the eye wearing device, and the mode will be illustrated in detail thereafter by referring to FIG. 6A, FIG. 6B and FIG. 6C.

FIG. 6A to FIG. 6C are schematic diagrams of a configuration mode of a reservoir and a microchannel in an eye wearing device according to the fourth embodiment of the disclosure, in which FIG. 6A is a top view schematic diagram, and FIG. 6B and FIG. 6C are straight cutting cross-section schematic diagrams along a tangent line D-D′ in FIG. 6A. Only FIG. 6B and FIG. 6C respectively represent different modes of a gradually reducing microchannel. FIG. 6B is a cross-section schematic diagram of a smooth gradually reducing mode. FIG. 6C is a cross-section schematic diagram of a stepped gradually reducing mode. The fourth embodiment in FIG. 6A, FIG. 6B and FIG. 6C is similar to the first embodiment in FIG. 1A, so that the specifications and the configuration of the identical assemblies are not repeated herein. It should be noted that, in order to clearly show the mode configuration of a reservoir 42 and microchannels 44, the drawing of an edge contour line of the eye wearing device is omitted in FIG. 6A, FIG. 6B and FIG. 6C, and only the reservoir 42 and the microchannels 44 are drawn. Additionally, only two microchannels 44 are drawn in this part to be connected to the reservoir 42, but the number of the microchannels 44 can still be adjusted according to practical requirements, and the disclosure is not limited thereto. For example, two, four, six, eight, ten and other number of microchannels 44 can be arranged to be connected to the reservoir 42.

Referring to FIG. 6A, FIG. 6B and FIG. 6C at the same time, the diameter of the microchannel 44 is gradually reduced from the edge of the eye wearing device to the reservoir 42 so as to enhance a drug filling and bubble eliminating mechanism promoted through capillary action. In the present embodiment, the diameter of the microchannel 44 can be gradually reduced from the edge of the eye wearing device in the cross-section direction of the eye wearing device to the reservoir 42. Therefore, in the top view schematic diagram of FIG. 6A, the diameters of the microchannel 44s are uniform. In the cross-section schematic diagrams of FIG. 6B and FIG. 6C, the diameter of the microchannel 44 has a gradually reducing trend from the edge of the eye wearing device to the reservoir 42. More specifically, through the top view schematic diagram of FIG. 6A, if an angle from one end, near the edge of the eye wearing device, of the microchannel 44 to the reservoir 42 is used as an inspection view angle, two side walls of the microchannels 44 are respectively defined as a left side wall 44L and a right side wall 44R. As shown in FIG. 6A, the distance between the left side wall 44L and the right side wall 44R of the microchannel 44 keeps unchanged. In the cross-section schematic diagrams of FIG. 6B and FIG. 6C, a lower side wall 44D of the microchannel 44 shows a trend of gradually approaching an upper side wall 44U. In other words, in the present embodiment, the width between the left side wall 44L and the right side wall 44R of the microchannel 44 keeps unchanged from the edge of the eye wearing device to the reservoir 42. The height between the upper side wall 44U and the lower side wall 44D of the microchannel 44 is gradually reduced from the edge of the eye wearing device to the reservoir 42. It should be noted that in the present embodiment, the way that the diameter of the microchannel 44 is gradually reduced from the edge of the eye wearing device to the reservoir 42 in a smooth and gradually reducing manner (as shown in FIG. 6B) or a step-like and gradually reducing manner (as shown in FIG. 6C), but the present disclosure provides no limitation to the gradually reducing manner.

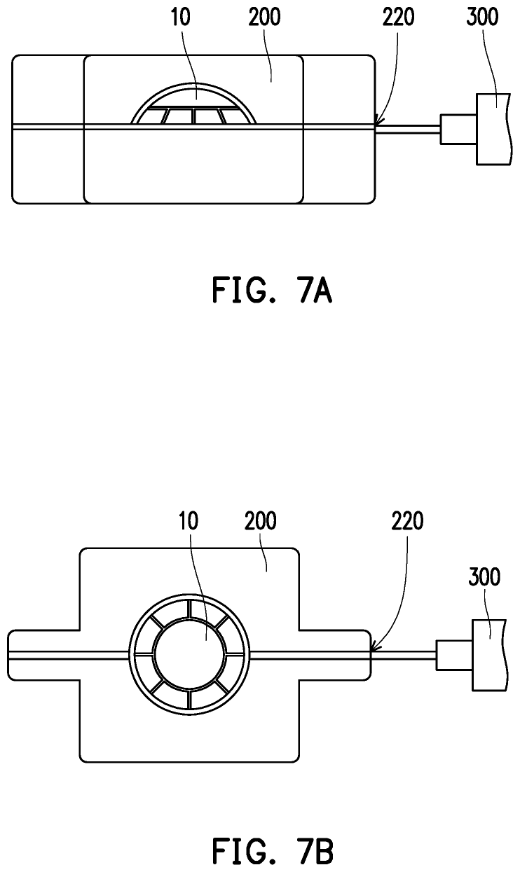

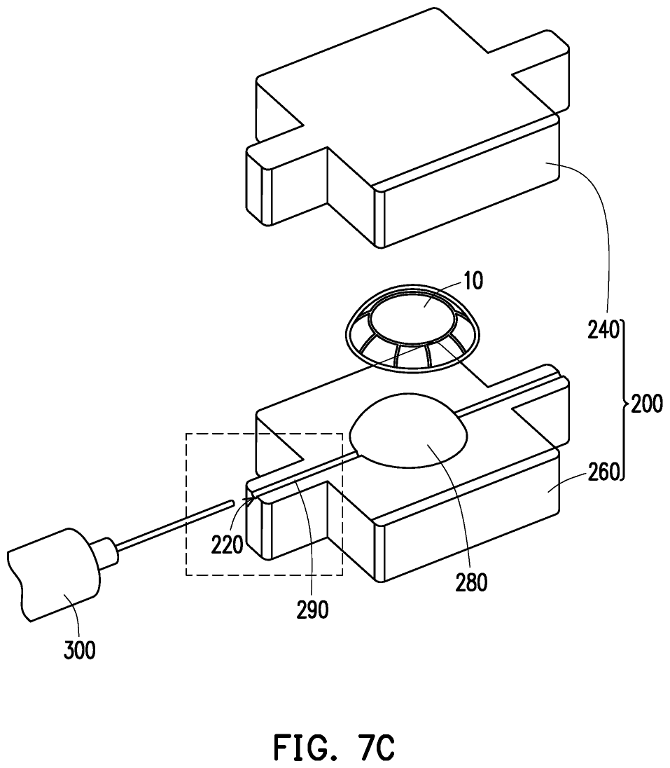

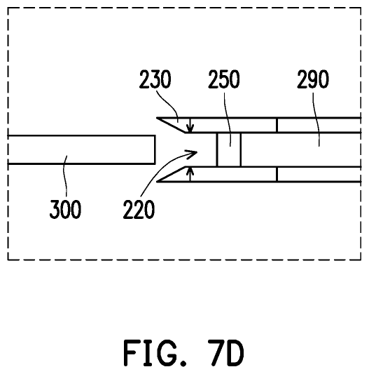

FIG. 7A to FIG. 7D are schematic diagrams of a package design of the eye wearing device according to the disclosure, in which FIG. 7A is a cross-section schematic diagram, FIG. 7B is a top view schematic diagram, FIG. 7C is a stereoscopic schematic diagram, and FIG. 7D is a local amplification schematic diagram.

Referring to FIG. 7A, FIG. 7B and FIG. 7C altogether, the eye wearing device 10 can be packaged in a hard packaging clamp 200 so that the eye wearing device 10 can be supported and is prevented from collapsing and deforming, the completeness of the microchannel can be maintained, and the drug filling precision can be improved. The eye wearing device 10 is used as an example in FIG. 7A, FIG. 7B and FIG. 7C, but the disclosure is not limited thereto. For example, eye wearing devices 20, 30A, 30B, 40A, 40B or 40C and the like can also be used. The packaging mode can use vacuum and negative pressure packaging modes, but is not limited thereto. The hard packaging clamp 200 can include a top cover 240 and a base 260. A protrusion 280 and at least one ditch 290 are arranged on the base 260. The protrusion 280 is connected to the ditch 290. The ditch 290 includes an injection opening 220 through which the drug is injected in. The average diameter of the cross section of the ditch can be about 50 μm to 1000 μm, for example, 100 μm to 950 μm, 150 μm to 900 μm, 200 μm to 850 μm, 250 μm to 800 μm, 300 μm to 750 μm, 350 μm to 700 μm, 500 μm to 750 μm, 500 μm to 800 μm, etc., but the disclosure is not limited thereto.

In the present embodiment, the top cover 240 includes a recessed part, the shape and the position of the recessed part correspond to those of the protrusion 280. The eye wearing device 10 can be put in the protrusion 280 of the base 260, in which the average curvature radius of the protrusion 280 is similar to that of the eye wearing device 10 so that the eye wearing device 10 is clamped in the protrusion 280 when the base 260 and the top cover 240 are buckled. The difference between the average curvature radius of the protrusion 280 and the average curvature radius of the eye wearing device 10 is only about 1 mm, 0.8 mm, 0.5 mm, 0.3 mm or 0.1 mm, etc., but the disclosure is not limited thereto.

Then, after the position of the eye wearing device 10 is fixed by the hard packaging clamp 200, drug filling can be performed by an injector 300 at the injection opening 220 of the hard packaging clamp 200, the drug can flow into the eye wearing device 10 on the protrusion 280 through the ditch 290 from the injection opening 220, and further enters the reservoir 12 through the microchannel 14 of the eye wearing device 10. In detail, as shown in FIG. 7D, a splay guide opening 230 can be further arranged in the position of the injection opening 220, so that the injector 300 is conveniently aligned with the injection opening 220. The injector 300 can include but is not limited to a needle head. For example, a dropper or a pipetman can be adopted for injecting the drug. Additionally, a sealing film 250 (or a rubber plug) can be arranged in front of or behind the guide opening 230 so as to maintain the vacuum or negative pressure in a package of the hard packaging clamp 200. When the injector 300 is inserted into the injection opening 220, the sealing film 250 can be fractured, and the drug filling is further performed in the above-mentioned mode.

Then, in FIG. 7A, FIG. 7B and FIG. 7C, only two ditches 290 are drawn, but the required ditches can be practically arranged according to requirements, for example, four, six, eight and so on, but the disclosure is not limited thereto. Additionally, different effective doses of drugs can be further injected through different injection openings, so that different kinds of required drugs are loaded in different reservoirs of the eye wearing device. For example, the eye wearing device 20 can be used as a drug loading object to be put in the protrusion 280 of the hard packaging clamp 200, one microchannel 24 respectively arranged in different reservoirs 22 faces one ditch 290, so that different drugs are delivered into the corresponding microchannel 24 by different ditches 290, and different drugs are loaded in different reservoirs 22 of the eye wearing device 20.

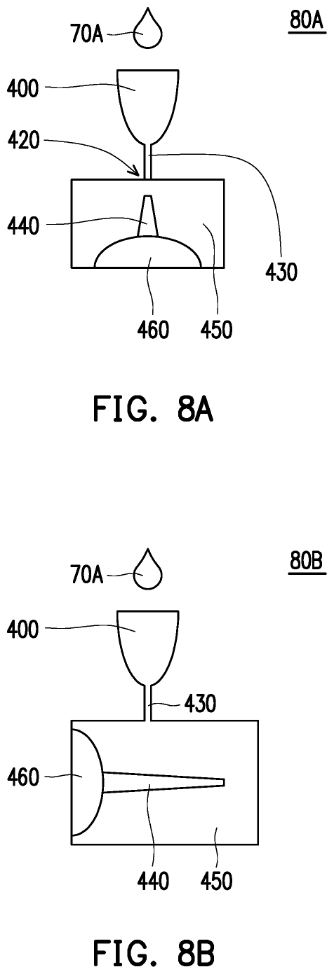

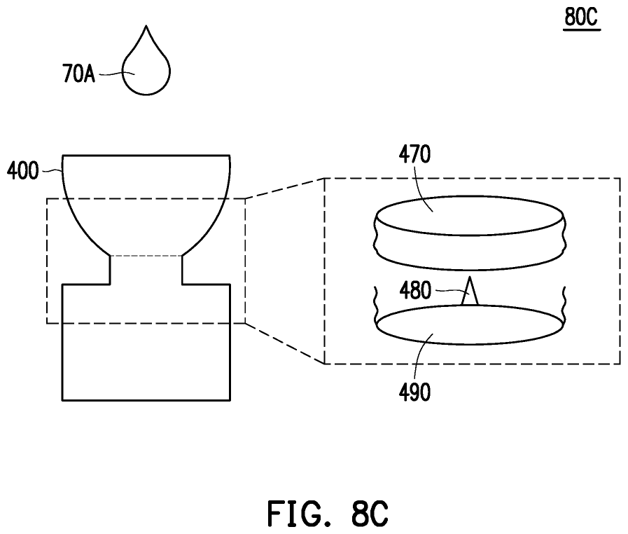

FIG. 8A, FIG. 8B and FIG. 8C are schematic diagrams of another package design and use method of the eye wearing device according to the disclosure.

In addition to the package design and use method of the eye wearing device illustrated in FIG. 7A, FIG. 7B and FIG. 7C, in order to avoid the filling by injection, the package designs shown in FIG. 8A, FIG. 8B and FIG. 8C can also be used. For the package designs in FIG. 8A, FIG. 8B and FIG. 8C, a drug drop container is used as a main device, which can replace the injector 300 in FIG. 7A, FIG. 7B and FIG. 7C and can be matched with the hard packaging clamp 200 in FIG. 7A, FIG. 7B and FIG. 7C to be used so as to fill effective doses of drugs into the eye wearing device 10. Referring to FIG. 8A, a drug drop container 80A can include a containing part 400, a sealing film 420, a safe invisible needle 440 and a storage space 450, in which the storage space 450 is arranged below the sealing film 420, and the safe invisible needle 440 can be arranged in the storage space 450. A connecting pipe 430 is arranged between the containing part 400 and the sealing film 420, one end of the connecting pipe 430 is connected to the bottom of the containing part 400, and the other end of the connecting pipe 430 is provided with the sealing film 420 configured to seal the opening. Additionally, a soft pad 460 can be arranged at the bottom of the safe invisible needle 440 in the storage space 450, for example, a plastic soft pad, so that the operation of the pressing action of the safe invisible needle 440 is convenient. The soft pad is favourable for the rebounding after the pressing action, so that the condition of hindering the drug from flowing to the storage space 450 from the connecting pipe 430 is avoided.

In operation, firstly, a drug 70A can be dropped into the containing part 400. After a user drops the drug 70A into the containing part 400 according to recommended doses, the soft pad 460 below the safe invisible needle 440 is pressed in a direction towards the sealing film 420 so that the safe invisible needle 440 pierces the sealing film 420, an effective dose of the drug 70A overflow into the storage space 450 through the connecting pipe 430 from the containing part 400. Then, referring to FIG. 8A and FIG. 7C at the same time, the drug 70A in the storage space 450 can be injected into the injection opening 220 of the hard packaging clamp 200 connected to the storage space 450 to enter the ditch 290 of the hard packaging clamp 200. The volume of the storage space 450 is smaller than that of the containing part 400, but is much greater than that of the reservoir 12 and the microchannel 14 in the eye wearing device 10, so that the problem of not filling up drug cannot occur.

Referring to FIG. 8B, a structure and an operation method of FIG. 8B are similar to those of FIG. 8A. The difference is that no sealing film 420 is arranged in a drug drop container 80B. In other words, the connecting part 430 is communicated with the storage space 450. The soft pad 460 can be arranged at a side surface. Meanwhile, the safe invisible needle 440 steers. In operation, the drug 70A is firstly dropped into the containing part 400 according to the recommended doses. Then, referring to FIG. 8B, FIG. 7C and FIG. 7D at the same time, the soft pad 460 is pressed in a direction towards the injection opening 220 of the hard packaging clamp 200, so that the safe invisible needle 440 can pierce the sealing film 250 of the injection opening 220, the effective dose of the drug 70A can overflow into the storage space 450 through the connecting part 430 from the containing part 400 through negative pressure, and is then injected into the injection opening 220 of the hard packaging clamp 200 connected to the storage space 450 through the storage space 450 and then enters the ditch 290 of the hard packaging clamp 200.

Referring to FIG. 8C, a structure and an operation method in FIG. 8C are similar to those in FIG. 8A. The difference is that in a drug drop container 80C, the connecting pipe 430, the sealing film 420, the safe invisible needle 440 and the soft pad 460 in FIG. 8A are replaced by a screw cap 470 with a sealing film and a support element 490 with a sharp object 480 (such as the safe invisible needle, a scraper or other sharp articles capable of being used for damaging the sealing film). In operation, the drug 70A is dropped into the containing part 400 firstly in the same way according to the recommended doses. Then, referring to FIG. 8C and FIG. 7C at the same time, the screw cap 470 is downwards screwed so that the sharp object 480 damages the sealing film in the screw cap 470, and the effective dose of the drug overflows into the storage space below the containing part 400 through a damage opening, and is then injected into the injection opening 220 of the hard packaging clamp 200 connected to the storage space through the storage space and then enters the ditch 290 of the hard packaging clamp 200.

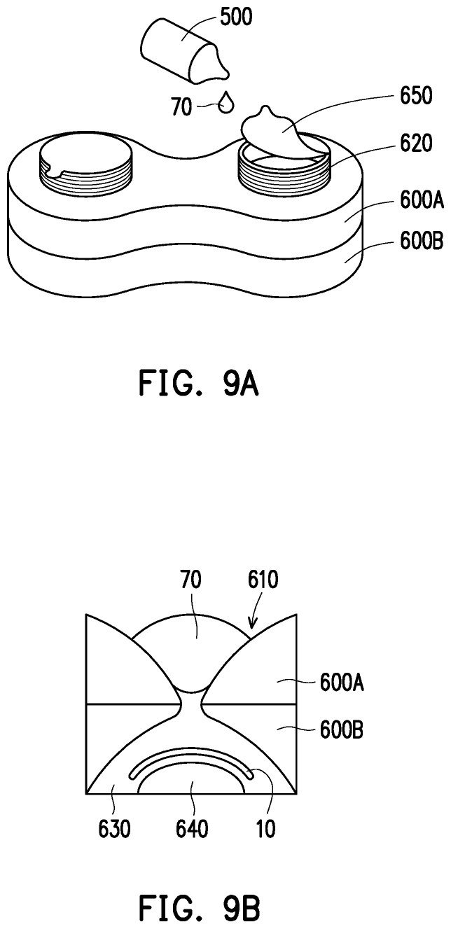

FIG. 9A and FIG. 9B are schematic diagrams of yet another package design and use method of the eye wearing device according to the disclosure, in which FIG. 9A is a stereoscopic schematic diagram, and FIG. 9B is a cross-section schematic diagram.

Structures in FIG. 9A and FIG. 9B are similar to those of hard packaging clamps 200 in FIG. 7A, FIG. 7B and FIG. 7C. The difference is that the structure is designed in a form like a contact lenses box, and is used by being preferably matched with the drug drop container 80C in FIG. 8C. Referring to FIG. 9A and FIG. 9B at the same time, an isolation film 650 configured to prevent pollution is torn away at first, then, the drug 70 is dropped into a drug drop container 620 according to the recommended doses by using a drop tool 500, and the drug drop container 620 here is similar to the drug drop container 80C in FIG. 8C. The drug drop container 620 is rotated so that a drug containing space 610 positioned in a top cover 600A is disconnected from a storage space 630 positioned in a base 600B, the drug 70 flows into the storage space 630 of the base 600B through the drug containing space 610 of the top cover 600A. A protrusion 640 is arranged in the storage space 630, the eye wearing device 10 can be put over the protrusion 640 of the storage space 630, and the average curvature radius of the protrusion 640 is similar to the average curvature radius of the eye wearing device 10. Therefore, the drug 70 in the storage space 630 can be filled into the eye wearing device 10.

In order to prove that the eye wearing device of the disclosure can effectively and slowly release a treatment drug, practical tests are respectively performed by aiming at the eye wearing devices of different modes designed by the disclosure hereafter, comparison to the existing drug-impregnated contact lenses is further performed, and operation and results of the experiment are shown hereafter in details.

The experiments are performed by aiming at the existing drug-impregnated contact lenses (Comparative example 1), the eye wearing device (Experimental example 1) in FIG. 1A, the eye wearing device similar to the eye wearing device in FIG. 1A but only provided with two channels (Experimental example 2, six openings of the eye wearing device in FIG. 1A are sealed by adhesive tapes), the eye wearing device in FIG. 6C (Experimental example 3) and the eye wearing device in FIG. 5A (Experimental example 4), and release constant and release time of 90% of the drugs of each comparative example and each experimental example are tested.

An experimental method is shown as follows: a microchannel prototype product is made of PDMS and hydrophilic treatment is performed. Then, pure water is used for cleaning the prototype product and ventilation and drying are performed. The PDMS prototype product sucking a drug solution is slowly put into 10 mL of pure water to prevent from perturbance. After that, the room temperature is maintained and sampling is performed every hour to every several hours, in which the longest sampling time reaches three days, and the volume of each sampling is 150 μL. During the sampling, the water is taken while stirring is slowly performed, and a hole passage opening is avoided as well as the time is recorded. Later, the light absorbance value of the drug is read by a UV/VIS spectrophotometer, and a standard drug concentration curve is made for concentration comparison. The test results are shown as Table 1.

As can be seen from Table 1, compared with the existing drug-impregnated contact lenses (Comparative example 1), the eye wearing devices (Experimental example 1, Experimental example 2, Experimental example 3 and Experimental example 4) designed according to the disclosure can effectively and slowly release the treatment drug.

TABLE 1

| | Comparative | Experimental | Experimental | Experimental | Experimental |
|---|---|---|---|---|---|
| | example 1 | example 1 | example 2 | example 3 | example 4 |
| Release constant | 38.4 | 14 | 3 | 3.6 | 1.9 |
| Release time of | 1 | 2.5 | 12 | 7.5 | 20 |
| 90% of drugs (hr) | | | | | |

Although the disclosure is described with reference to the above embodiments, the embodiments are not intended to limit the disclosure. A person of ordinary skill in the art may make variations and modifications without departing from the spirit and scope of the disclosure. Therefore, the protection scope of the disclosure should be subject to the appended claims.

What is claimed is:

1. An eye wearing device, comprising:
a body, having a first surface, a second surface, a center, a first outer edge of the first surface and a second outer edge of the second surface;
at least one reservoir, arranged in the body and configured to load a drug; and
at least one microchannel, arranged close to the first outer edge of the first surface and the second outer edge of the second surface, wherein one end of the microchannel is connected to the reservoir so as to fill the drug into the reservoir, and the other end of the microchannel is an opening facing an edge of the eye wearing device, and configured to be in contact with a cornea and/or a sclera, and the opening is connected to the first outer edge of the first surface and the second outer edge of the second surface,
wherein an average diameter of a cross section of the microchannel is smaller than or equal to an average diameter of a cross section of the reservoir, an average curvature radius of the eye wearing device is 6 mm to 15 mm, and the eye wearing device is worn on the cornea and/or the sclera of a user through the second surface.

2. The eye wearing device according to claim 1, wherein the reservoir has the width of 30 μm to 5 mm, the height of 10 μm to 200 μm, the total volume of 0.002 μL to 20 μL, and an average diameter of a cross section of the microchannel is 20 μm to 150 μm.

3. The eye wearing device according to claim 1, wherein a diameter of the eye wearing device is 12 mm to 20 mm, and an average thickness of the eye wearing device is 20 μm to 400 μm.

4. The eye wearing device according to claim 1, wherein a diameter of the microchannel is gradually reduced from the edge of the eye wearing device to the reservoir.

5. The eye wearing device according to claim 1, wherein a diameter of one end of the microchannel connected to the reservoir is 10 μm to 200 μm, and a diameter of another end of the microchannel in contact with the cornea and/or the sclera is 40 μm to 200 μm.

6. The eye wearing device according to claim 1, wherein a lubricating material is coated on a surface of the eye wearing device.

7. The eye wearing device according to claim 6, wherein the lubricating material comprises mucoprotein, polyethylenimine, polyethylene glycol, polyacrylic acid, polymethacrylic acid, polyitaconic acid, polymaleic acid, carboxymethyl cellulose (CMC), hydroxypropyl methylcellulose (HPMC), polyvinylpyrrolidone, polyacrylamide, poleyvinylalcohol, hyaluronic acid, dextran, poly 2-hydroxyethyl methacrylate (poly HEMA), poly sulfonates, polylactate, urea, phosphoryl choline or a combination thereof.

8. The eye wearing device according to claim 7, wherein the lubricating material further comprises hydrophilic polypeptides, and amino acid in an amount of 75% or more by weight of the hydrophilic polypeptides is selected from a group consisting of aspartic acid (Asp or D), glutamic acid (Glu or E), histidine (His or H), lysine (Lys or K), asparagine (Asn or N), glutamine (Gln or Q), arginine (Arg or R), serine (Ser or S), threonine (Thr or T) and tyrosine (Tyr or Y).

9. The eye wearing device according to claim 1, wherein the material of the eye wearing device comprises bio-derived polymers, non-bio-derived polymers or a combination thereof.

10. The eye wearing device according to claim 9, wherein the bio-derived polymers comprise collagen, gelatin, chitin, cellulose or a combination thereof.

11. The eye wearing device according to claim 9, wherein the non-bio-derived polymers comprise polyethylene glycol (PEG), propylene glycol diacrylate (PPGDA), polydimethylsiloxane (PDMS), poly(methyl methacrylate) (PMMA), poly(hydroxyethyl methacrylate) (PHEMA) or a combination thereof.

12. The eye wearing device according to claim 1, further comprising a package for loading the eye wearing device.

13. The eye wearing device according to claim 12, wherein the package comprises a hard packaging clamp, and the hard packaging clamp comprises:
a base, wherein a protrusion and at least one ditch are arranged on the base, the protrusion is connected to the ditch, and the protrusion is configured to place the eye packaging device, and the ditch has an injection opening for injection of the drug; and
a top cover, having a recessed part, wherein the shape and the position of the recessed part correspond to those of the protrusion.

14. The eye wearing device according to claim 13, wherein an average curvature radius of the protrusion is similar to an average curvature radius of the eye wearing device, so that the eye wearing device is clamped in the protrusion when the base and the top cover are buckled.

15. The eye wearing device according to claim 13, wherein an average diameter of a cross section of the ditch is 50 μm to 1000 μm.

16. The eye wearing device according to claim 13, wherein the injection opening further comprises a guide opening configured for aligned injection.

17. The eye wearing device according to claim 13, wherein the package further comprises:
at least one drug drop container configured to load an effective dose of the drug.

18. The eye wearing device according to claim 17, wherein the drug drop container comprises:
a containing part;
a connecting pipe, connected to the bottom of the containing part;
a storage space, arranged below the connecting part and connected to the injection opening of the hard packaging clamp; and
a safe invisible needle, arranged in the storage space.

19. The eye wearing device according to claim 18, wherein a sealing film is arranged between the storage space and the connecting pipe, and the drug overflows into the storage space through the connecting pipe from the containing part when the safe invisible needle is pressed, and is then injected into the injection opening from the storage space to enter the ditch of the hard packaging clamp.

20. The eye wearing device according to claim 17, wherein the drug drop container comprises:
a screw cap, configured with a sealing film;
a support element, configured with a sharp object; and
a storage space, arranged below the support element and connected to the injection opening of the hard packaging clamp,
wherein after the screw cap is downwards screwed, the sharp object damages the sealing film in the screw cap, so that the drug overflows into the storage space and is then injected into the injection opening from the storage space to enter the ditch of the hard packaging clamp.
